(19) United States
(12) Patent Application Publication
(10) Pub. No.: US 2016/0159715 A1
(43) Pub. Date: Jun. 9, 2016

(54) PROCESS FOR PRODUCING 1,6-HEXANEDIOL

(71) Applicant: E I DU PONT DE NEMOURS AND COMPANY, Wilmington, DE (US)

(72) Inventors: RONALD JAMES DAVIS, CHRISTIANA, PA (US); CARL ANDREW MENNING, NEWARK, DE (US); JOSEPH E. MURPHY, WOODBURY, NJ (US); JOACHIM C. RITTER, WILMINGTON, DE (US); SOURAV KUMAR SENGUPTA, WILMINGTON, DE (US)

(21) Appl. No.: 14/956,425

(22) Filed: Dec. 2, 2015

Related U.S. Application Data
(60) Provisional application No. 62/088,037, filed on Dec. 5, 2014.

(51) Int. Cl. C07C 29/48

(52) U.S. Cl. CPC C07C 29/48

(57) ABSTRACT

Disclosed herein are processes for producing 1,6-hexanediol. In one embodiment, the process comprises a step of contacting 3,4-dihydro-2H-pyran-2-carbaldehyde, a solvent, and hydrogen in the presence of a catalyst at a reaction temperature between about 0° C. and about 120° C. at a pressure and for a reaction time sufficient to form a product mixture comprising 1,6-hexanediol. In one embodiment, the catalyst comprises a metal M1, a metal M2 or an oxide of M2, and a support, wherein M1 is Rh, Ir, Ni, Pd, or Pt, and M2 is Mo, W, or Re; or M1 is Cu and M2 is Ni, Mn, or W.

### FIELD OF DISCLOSURE

Processes for producing 1,6-hexanediol from acrolein dimer are provided.

### BACKGROUND

Alpha, omega-diols such as 1,6-hexanediol are useful as chemical intermediates for the production of agrichemicals, pharmaceuticals, and polymers. For example, a,w-diols can be used as plasticizers and as comonomers in polyesters and polyether-urethanes. 1,6-Hexanediol is a useful intermediate in the industrial preparation of nylon 66. 1,6-Hexanediol can be converted by known methods to 1,6-hexamethylene diamine, a starting component in nylon production.

There is an existing need for new routes to 1,6-hexanediol which utilize inexpensive feedstocks and which can offer cost advantages over alternative methods of 1,6-hexanediol production.

### SUMMARY

In one embodiment, a process is provided, the process comprising the step:

contacting 3,4-dihydro-2H-pyran-2-carbaldehyde, a solvent, and hydrogen in the presence of a catalyst at a reaction temperature between about 0° C. and about 120° C. at a pressure and for a reaction time sufficient to form a product mixture comprising 1,6-hexanediol.

In one embodiment, the solvent comprises an alcohol, an ether, an ester, an aromatic hydrocarbon, an aliphatic hydrocarbon, or mixtures thereof. In one embodiment, the solvent is miscible with water and further comprises from about 0 weight percent to about 75 weight percent water, based on the total weight of water and solvent.

In one embodiment, the catalyst comprises a metal M1, a metal M2 or an oxide of M2, and a support, wherein M1 is Rh, Ir, Ni, Pd, or Pt, and M2 is Mo, W, or Re; or M1 is Cu and M2 is Ni, Mn, or W. In one embodiment, M1 is Cu and M2 is Ni, Mn, or W. In one embodiment, M1 is Rh, Ir, Ni, Pd, or Pt, and M2 is Mo, W, or Re. In one embodiment, M1 is Pt and M2 is W. In one embodiment, the support comprises WO₃, V₂O₅, MoO₃, SiO₂, Al₂O₃, TiO₂, ZrO₂, tungstated ZrO₂, SiO₂—Al₂O₃, SiO₂—TiO₂, montmorillonite, zeolites, or mixtures thereof. In one embodiment, M1 is Pt, M2 is W, and the support comprises TiO₂.

In one embodiment, the pressure is between about 690 kPa and about 6895 kPa. In one embodiment, the concentration of 3,4-dihydro-2H-pyran-2-carbaldehyde in the solvent is between about 1 wt % and about 60 wt %, based on the total weight of 3,4-dihydro-2H-pyran-2-carbaldehyde and solvent. In one embodiment, the contacting step comprises a first step of contacting the solvent and hydrogen in the presence of the catalyst to form an initial mixture, and a second step of adding the 3,4-dihydro-2H-pyran-2-carbaldehyde to the initial mixture. In one embodiment, the contacting is performed in a continuous manner. In one embodiment, the contacting is performed in a batch manner.

In one embodiment, the product mixture further comprises tetrahydro-2H-pyran-2-methanol. In one embodiment, the product mixture further comprises 1,2,6-hexanetriol. In one embodiment, the product mixture further comprises 1-hexanol. In one embodiment, the process further comprises a step of separating at least a portion of the 1,6-hexanediol from the product mixture. In one embodiment, the 3,4-dihydro-2H-pyran-2-carbaldehyde is obtained from dimerization of acrolein.

In one embodiment, the product mixture further comprises tetrahydro-2H-pyran-2-methanol or 1,2,6-hexanetriol, and the process further comprises a step of: reacting the product mixture with hydrogen in the presence of the catalyst at a second temperature between about 120° C. and about 260° C. at a second pressure of about 5515 kPa to about 13,800 kPa to form a second product mixture enriched in 1,6-hexanediol. In one embodiment, the catalyst comprises a metal M1, a metal M2 or an oxide of M2, and a support, wherein M1 is Rh, Ir, Ni, Pd, or Pt, and M2 is Mo, W, or Re. In one embodiment, the process further comprises a step of separating at least a portion of the 1,6-hexanediol from the second product mixture.

In some embodiments, the process further comprises the steps:

(a) optionally, isolating at least a portion of the 1,6-hexanediol from the product mixture or second product mixture;

(b) contacting the 1,6-hexanediol with ammonia and hydrogen in the presence of a reductive amination catalyst at a temperature and for a time sufficient to form an amination product mixture comprising 1,6-diaminohexane; and

(c) optionally, isolating at least a portion of the 1,6-diaminohexane from the amination product mixture

### DETAILED DESCRIPTION

As used herein, where the indefinite article “a” or “an” is used with respect to a statement or description of the presence of a step in a process disclosed herein, it is to be understood, unless the statement or description explicitly provides to the contrary, that the use of such indefinite article does not limit the presence of the step in the process to one in number.

As used herein, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope be limited to the specific values recited when defining a range.

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the term “about” modifying the quantity of an ingredient or reactant employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. The term “about” may mean within 10% of the reported numerical value, preferably within 5% of the reported numerical value.

As used herein, the abbreviation “16HD” refers to 1,6-hexanediol. The chemical structure of 1,6-hexanediol is represented by Formula (I).

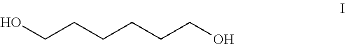

As used herein, the abbreviation “126HT” refers to 1,2,6-hexanetriol. The chemical structure of 1,2,6-hexanetriol is represented by Formula (II).

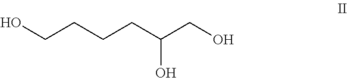

As used herein, the abbreviation “ACD” refers to “acrolein dimer”, also known as 3,4-dihydro-2H-pyran-2-carbaldehyde or 3,4-dihydro-2H-pyran-2-carboxaldehyde. The chemical structure of acrolein dimer is represented by Formula (III).

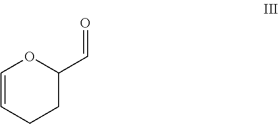

As used herein, the abbreviation “DHPM” refers to 3,4-dihydro-2H-pyran-2-methanol, the chemical structure of which is represented by Formula (IV).

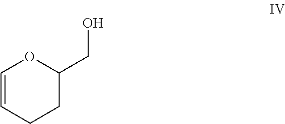

As used herein, the abbreviation “THPM” refers to tetrahydro-2H-pyran-2-methanol, also known as 2-hydroxymethyltetrahydropyran, and includes a racemic mixture of isomers. The chemical structure of tetrahydro-2H-pyran-2-methanol is represented by Formula (V).

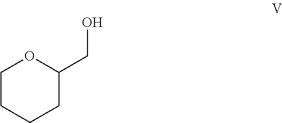

As used herein, the abbreviation “THPC” refers to tetrahydro-2H-pyran-2-carbaldehyde, the chemical structure of which is represented by Formula (VI).

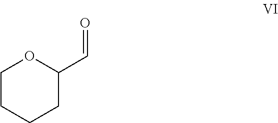

In one embodiment, a process is provided, the process comprising the step: contacting 3,4-dihydro-2H-pyran-2-carbaldehyde, a solvent, and hydrogen in the presence of a catalyst at a reaction temperature between about 0° C. and about 100° C. at a pressure and for a reaction time sufficient to form a product mixture comprising 1,6-hexanediol. The product mixture may further comprise one or both of tetrahydro-2H-pyran-2-methanol or 1,2,6-hexanetriol, as shown below in Scheme I. In one embodiment, the process further comprises a step of separating at least a portion of the 1,6-hexanediol from the product mixture.

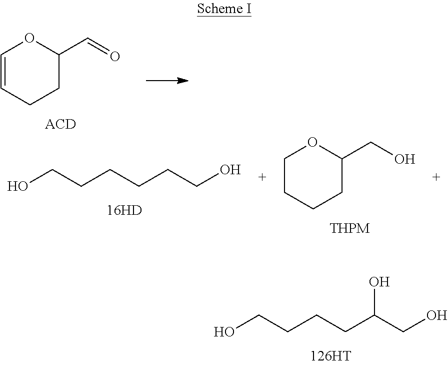

In one embodiment, the product mixture further comprises tetrahydro-2H-pyran-2-methanol or 1,2,6-hexanetriol, and the process further comprises the step of reacting the product mixture with hydrogen in the presence of the catalyst at a second temperature between about 120° C. and about 260° C. at a second pressure of about 800 psi to about 2000 psi to form a second product mixture enriched in 1,6-hexanediol, as shown below in Scheme II. In one embodiment, the process further comprises a step of separating at least a portion of the 1,6-hexanediol from the second product mixture.

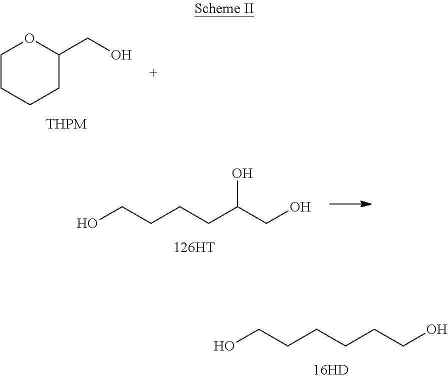

3,4-Dihydro-2H-pyran-2-carbaldehyde can be obtained commercially or prepared by methods known in the art. In one embodiment, 3,4-dihydro-2H-pyran 2-carbaldehyde can be obtained from the dimerization of acrolein, also referred to as propenal, for example as disclosed in U.S. Pat. No. 2,479,284. Acrolein, in turn, may be derived from inexpensive feedstocks such as glycerin, for example as disclosed in U.S. Pat. No. 7,847,131. The use of 3,4-dihydro-2H-pyran-2-carbaldehyde as a starting material for 1,6-hexanediol production offers an alternative to other methods, such as the hydrogenation of adipic acid.

In one embodiment, the catalyst comprises a metal M1, a metal M2 or an oxide of M2, and a support, wherein:

M1 is Rh, Ir, Ni, Pd, or Pt, and M2 is Mo, W, or Re; or

M1 is Cu and M2 is Ni, Mn, or W.

In one embodiment, the catalyst comprises a metal M1, a metal M2 or an oxide of M2, and a support, wherein:

M1 is Rh, Ir, Ni, Pd, or Pt, and M2 is Mo, W, or Re.

In one embodiment, the catalyst comprises a metal M1, a metal M2 or an oxide of M2, and a support, wherein:

M1 is Cu and M2 is Ni, Mn, or W.

In one embodiment, the catalyst comprises metals M1 and M2, and a support, wherein M1 is Pt and M2 is W; or M1 is Pt and M2 is Mo; or M1 is Pt and M2 is Re; or M1 is Rh and M2 is W; or M1 is Rh and M2 is Mo; or M1 is Rh and M2 is Re; or M1 is Ir and M2 is Mo; or M1 is Ir and M2 is W; or M1 is Ir and M2 is Re; or M1 is Ni and M2 is W; or M1 is Pd and M2 is Mo; or M1 is Pd and M2 is W; or M1 is Pd and M2 is Re; or M1 is Ni and M2 is Mo; or M1 is Ni and M2 is Re; or M1 is Cu and M2 is W; or M1 is Cu and M2 is Ni; or M1 is Cu and M2 is Mn.

The catalysts utilized in the processes described herein can be synthesized by any conventional method for preparing catalysts, for example, deposition of metal salts from aqueous or organic solvent solutions via impregnation or incipient wetness, precipitation of an M1 component and/or an M2 component, or solid state synthesis. Preparation may comprise drying catalyst materials under elevated temperatures from 30-250° C., preferably 50-150° C.; calcination by heating in the presence of air at temperatures from 250-800° C., preferably 300-450° C. ; and reduction in the presence of hydrogen at 100-400° C., preferably 200-300° C., or reduction with alternative reducing agents such as hydrazine, formic acid or ammonium formate. The above techniques may be utilized with powdered or formed particulate catalyst materials prepared by tableting, extrusion or other techniques common for catalyst synthesis. Where powdered catalysts materials are utilized, it will be appreciated that the catalyst support or the resulting catalyst material may be sieved to a desired particle size and that the particle size may be optimized to enhance catalyst performance.

The M1 and M2 components of the catalyst may be derived from any appropriate metal compound. Examples include but are not limited to: rhodium (III) chloride hydrate, copper (II) nitrate hydrate, nickel (II) chloride hexahydrate, iridium (IV) chloride hydrate, tetraammineplatinum (II) nitrate, platinum chloride, hexachloroplatinic acid, tetrachloroplatinic acid, palladium chloride, palladium nitrate, palladium acetate, iridium trichloride, ammonium perrhenate, ammonium tungsten oxide hydrate, ammonium molybdate hydrate, and manganese (II) nitrate hydrate. An example of a useful catalyst synthesis method is disclosed in the Experimental Section herein below.

The loading of M1 may be 0.1-50% by weight, for example 0.5-10% or 0.5-5% by weight, based on the weight of the prepared catalyst (i.e., including the catalyst support present). The loading of M2 may be 0.1-99.9% by weight, for example 2-10% by weight. In some embodiments, the molar ratio of M1 to M2 may be in the range of 1:0.5 to 1:5. Optionally, M2 may be incorporated into the catalyst support or serve as the catalyst support, for example Pt supported on tungsten oxide or molybdenum oxide. Regarding the catalyst, all percentages are interpreted as weight percent relative to the weight of the prepared catalyst.

As used herein, the term “support” means a material which is a component of the catalyst and is used as part of catalyst preparation to anchor the metals M1 and M2, providing a surface for metals M1 and M2 to associate with. Examples of useful supports may include WO₃, SiO₂, Al₂O₃, carbon, SiC, TiO₂, ZrO₂, SiO₂—Al₂O₃, clays such as montmorillonite, SiO₂—TiO₂, tungstated ZrO₂, V₂O₅, MoO₃, and zeolites such as H-Y, FAU (H-Y or USY), BEA (H-Beta), MFI (H-ZSM5), MEL (H-ZSM11) and MOR (H-Mordenite). Typically, tungstated ZrO₂ can comprise up to about 19 wt % W as WO₃ on ZrO₂, see for example S. Kuba et al in Journal of Catalysis, 216 (2003), p. 353-361. In one embodiment, the support comprises WO₃, V₂O₅, MoO₃, SiO₂, Al₂O₃, TiO₂, ZrO₂, tungstated ZrO₂, SiO₂—Al₂O₃, SiO₂—TiO₂, montmorillonite, zeolites, or mixtures thereof. In one embodiment, the support comprises TiO₂, ZrO₂, SiO₂, or mixtures thereof. In one embodiment, the support comprises TiO₂. In one embodiment, the support comprises ZrO₂. In one embodiment, the support comprises SiO₂.

The prepared catalyst may be in any physical form typical for heterogeneous catalysts, including but not limited to: powdered (also known as “fluidized”) forms with 0.01-150 μm particle size, formed tablets, extrudates, spheres, engineered particles having uniform 0.5-10 mm size, monolithic structures on which surfaces the catalyst is applied, or combinations of two or more of the above. It is desirable that M1 be intimately associated with the M2 component, as measured by transmission electron microscopy with energy dispersive spectroscopy. In some embodiments, the particle size of the M1 component may be less than 10 nm, for example less than 3 nm, as measured by the same techniques. In this case, particle size of the M1 component may be interpreted as particle size of a mixture of the M1 and M2 components, an alloy of the M1 and M2 components, a particle of the M1 component adjacent to a particle of the M2 component, or a particle of the M1 component on the support which contains the M2 component.

The catalyst may be present in any weight ratio to the substrate sufficient to catalyze the conversion to 1,6-hexanediol, generally in the range of 0.0001:1 to 1:1, for example in the range of 0.001:1 to 0.5:1 for batch reactions. For continuous reactions, the same ratios are appropriate where the weight ratio of feed to catalyst is defined as weight of substrate feed processed per weight of catalyst.

The contacting step is performed using a solvent, which may serve to reduce the viscosity of the system to improve fluidity of the catalyst in the reaction vessel, and/or to remove the heat of reaction and improve the performance of the process. The solvent may be present in a range from about 1% to 95% by weight of the total reaction mixture, excluding the catalyst. In some embodiments, the solvent may be present in a range from about 5% to about 95%, or from about 10% to about 90% by weight. Solvents useful in the present processes typically are those which dissolve the substrate 3,4-dihydro-2H-pyran-2-carbaldehyde and are substantially inert under the reaction conditions of the contacting step. In one embodiment, the solvent comprises an alcohol, an ether, an ester, an aromatic hydrocarbon, an aliphatic hydrocarbon, or mixtures thereof. As used herein, the term “mixtures thereof” encompasses both mixtures within and mixtures between solvent classes, for example mixtures of alcohols, and also mixtures between alcohols and ethers, for example.

In one embodiment, the solvent comprises an alcohol. Alcohols useful as solvent in the processes disclosed herein may be linear or branched, unsubstituted or substituted with alkyl groups or halides, and may contain from one to 15 carbon atoms. Examples of useful alcohols include methanol, ethanol, 1-propanol, 2-propanol, 2-methylpropanol, butanols, pentanols, hexanols, heptanols, and octanols.

In one embodiment, the solvent comprises an ether. Ethers useful as solvent in the processes disclosed herein may be linear or branched, unsubstituted or substituted with alkyl groups or halides, cyclic or acyclic, and may contain from two to 18 carbon atoms. Examples of useful ethers include tetrahydrofuran, tetrahydropyran, tetrahydro-2H-pyran-2-methanol, 1,4-dioxane, diethyl ether, dipropyl ether, dibutyl ether, dipentyl ether, and dihexyl ether.

In one embodiment, the solvent comprises an ester. Esters useful as solvent in the processes disclosed herein may be linear or branched, unsubstituted or substituted with alkyl groups or halides, and may contain from two to 18 carbon atoms. Examples of useful esters include methyl acetate, ethyl acetate, propyl acetate, butyl acetate, methyl butyrate, ethyl butyrate, butyl butyrate, methyl hexanoate, ethyl hexanoate, propyl hexanoate, butyl hexanoate, and hexyl hexanoate.

In one embodiment, the solvent comprises an aromatic hydrocarbon. Aromatic hydrocarbons useful as solvent in the processes disclosed herein may be unsubstituted or substituted with alkyl groups or halides, and may contain from six to 18 carbon atoms. Examples of useful aromatic hydrocarbons include benzene, toluene, o-xylene, m-xylene, p-xylene, trimethylbenzenes, and cumene.

In one embodiment, the solvent comprises an aliphatic hydrocarbon. Aliphatic hydrocarbons useful as solvent in the processes disclosed herein may be substituted or unsubstituted cycloalkanes or n-alkanes, and may contain from six to 18 carbon atoms. Examples of useful aliphatic hydrocarbons include cyclohexane, cyclooctane, isooctane, dodecane, tetradecane, hexadecane, and octadecane.

Suitable solvents are typically available commercially from various sources, such as Sigma-Aldrich (St. Louis, Mo.), in various grades, many of which may be suitable for use in the processes disclosed herein. Technical grades of a solvent can contain a mixture of compounds, including the desired component and higher and lower molecular weight components or isomers.

In one embodiment, the solvent is anhydrous, for example containing less than about 0.1 wt % water, based on the total weight of water and solvent. In one embodiment, the solvent is miscible with water and further comprises from about 0 weight percent to about 75 weight percent water, based on the total weight of water and solvent. In some embodiments, the water concentration in the solvent is between and optionally includes any two of the following values: 0 wt %, 0.01 wt %, 0.05 wt %, 0.1 wt %, 0.5 wt %, 1 wt %, 2 wt %, 3 wt %, 4 wt %, 5 wt %, 6 wt %, 7 wt %, 8 wt %, 9 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, and 75 wt %. In some embodiments, the water concentration in the solvent is between about 0 wt % and about 20 wt %. In some embodiments, the water concentration in the solvent is between about 0 wt % and about 5 wt %. Larger amounts of water in the solvent may decrease the yield of 1,6-hexanediol, and may increase the yield of by-product 1,2,6-hexanetriol. In one embodiment, selecting a desired amount of water in the solvent may be useful as a method for adjusting the relative ratios of 1,6-hexanediol and 1,2,6-hexanetriol in the product mixture. In the contacting step, the concentration of 3,4-dihydro-2H-pyran-2-carbaldehyde in the solvent can be between about 1 wt % and about 70 wt %, based on the total weight of 3,4-dihydro-2H-pyran-2-carbaldehyde and solvent. In one embodiment, the concentration of 3,4-dihydro-2H-pyran-2-carbaldehyde in the solvent is between and optionally includes any two of the following values: 1 wt %, 5 wt %, 10 wt %, 15 wt %, 20 wt %, 25 wt %, 30 wt %, 35 wt %, 40 wt %, 45 wt %, 50 wt %, 55 wt %, 60 wt %, 65 wt %, and 70 wt %. In one embodiment, the concentration of 3,4-dihydro-2H-pyran-2-carbaldehyde in the solvent is between about 1 wt % and about 60 wt %. In one embodiment, the concentration of 3,4-dihydro-2H-pyran-2-carbaldehyde in the solvent is between about 1 wt % and about 30 wt %. In one embodiment, the concentration of 3,4-dihydro-2H-pyran-2-carbaldehyde in the solvent is between about 10 wt % and about 30 wt %. The 3,4-dihydro-2H-pyran-2-carbaldehyde concentrations referred to herein may be initial concentrations, for example when the process is performed in a batch manner, or steady-state concentrations, for example when the process is performed in a continuous manner.

Contacting the 3,4-dihydro-2H-pyran-2-carbaldehyde with solvent and hydrogen in the presence of a catalyst may be performed at a temperature between about 0° C. and about 120° C. In one embodiment, the temperature is between about 20° C. and about 120° C. In some embodiments, the temperature is between and optionally includes any two of the following values: 0° C., 5° C., 10° C., 15° C., 20° C., 25° C., 30° C., 35° C., 40° C., 45° C., 50° C., 55° C., 60° C., 65° C., 70° C., 75° C., 80° C., 85° C., 90° C., 95° C., 100° C., 105° C., 110° C., 115° C. and 120° C. In some embodiments, higher temperatures may also be used.

Contacting the 3,4-dihydro-2H-pyran-2-carbaldehyde substrate, solvent, and hydrogen in the presence of a catalyst may be performed at a reaction pressure between about 80 psi (550 kPa) and about 1015 psi (7000 kPa), for example between about 690 kPa and about 6985 kPa. Higher reaction pressures may also be used. The mole ratio of hydrogen to substrate is not critical as long as sufficient hydrogen is present to produce the desired 1,6-hexanediol. Hydrogen is preferably used in excess, and may optionally be used in combination with an inert gas such as nitrogen or argon. If an inert gas is used in combination with the hydrogen, the amount of the inert gas should be such that it does not negatively impact the formation of the product mixture. In some embodiments, the pressure of the contacting step is between and optionally includes any two of the following values: 550 kPa, 690 kPa, 800 kPa, 900 kPa, 1000 kPa, 1500 kPa, 2000 kPa, 2500 kPa, 3000 kPa, 3500 kPa, 4000 kPa, 4500 kPa, 5000 kPa, 5500 kPa, 6000 kPa, 6500 kPa, and 7000 kPa. The choice of operating pressure may be related to the reaction temperature and is often influenced by economic considerations and/or ease of operation.

To improve the yield of 1,6-hexanediol, the contacting step may be performed as two sequential steps. In one embodiment, the contacting step comprises a first step of contacting the solvent and hydrogen in the presence of the catalyst to form an initial mixture, and a second step of adding the 3,4-dihydro-2H-pyran-2-carbaldehyde to the initial mixture. The 3,4-dihydro-2H-pyran-2-carbaldehyde may be added all at once, in portions, or continuously.

Sufficient reaction time, in conjunction with a reaction temperature and pressure as disclosed herein above, enables formation of a product mixture comprising 1,6-hexanediol. The product mixture may further comprise tetrahydro-2H-pyran-2-methanol or 1,2,6-hexanetriol. In one embodiment, the product mixture further comprises tetrahydro-2H-pyran-2-methanol. In one embodiment, the product mixture further comprises 1,2,6-hexanetriol. In one embodiment, the product mixture further comprises 1-hexanol. In one embodiment, the product mixture comprises 1,6-hexanediol and 1-hexanol. The reaction products may be separated or purified by any common methods known in the art including distillation, wiped film evaporation, chromatography, adsorption, crystallization, and membrane separation.

Tetrahydro-2H-pyran-2-methanol and 1,2,6-hexanetriol can each be converted to 1,6-hexanediol under appropriate reaction conditions. Thus, in cases where the product mixture contains one or both of these compounds, the yield of 1,6-hexanediol may be increased by performing a second reaction step, using the same or different catalyst as for the conversion of 3,4-dihydro-2H-pyran-2-carbaldehyde to 1,6-hexanediol. In one embodiment, the product mixture further comprises tetrahydro-2H-pyran-2-methanol or 1,2,6-hexanetriol, and the process further comprises a step of reacting the product mixture with hydrogen in the presence of the catalyst at a second temperature between about 120° C. and about 260° C. at a second pressure of about 800 psi (5515 kPa) to about 2000 psi (13,800 kPa) to form a second product mixture enriched in 1,6-hexanediol. By “enriched in 1,6-hexanediol” is meant that the amount of 1,6-hexanediol, on a molar basis, is greater in the second product mixture than in the product mixture used as the feed material.

In one embodiment, the catalyst comprises a metal M1, a metal M2 or an oxide of M2, and a support, wherein M1 is Rh, Pt, or Ir; and M2 is Mo, W, or Re. The support may comprise WO₃, V₂O₅, MoO₃, SiO₂, Al₂O₃, TiO₂, ZrO₂, tungstated ZrO₂, SiO₂—Al₂O₃, SiO₂—TiO₂, montmorillonite, zeolites, or mixtures thereof, as disclosed herein above. In some embodiments, the product mixture may be reacted with hydrogen in the presence of the catalyst at a second temperature between and optionally including any two of the following values: 120° C., 130° C., 140° C., 150° C., 160° C., 170° C., 180° C., 190° C., 200° C., 210° C., 220° C., 230° C., 240° C., 250° C., and 260° C. In some embodiments, the second pressure is between and optionally includes any two of the following values: 5515 kPa, 6000 kPa, 7000 kPa, 8000 kPa, 9000 kPa, 10,000 kPa, 11,000 kPa, 12,000 kPa, 13,000 kPa, and 13,800 kPa. Optionally, the process may further comprise a step of separating at least a portion of the 1,6-hexanediol from the second product mixture.

The processes disclosed herein provide a useful synthetic route to 1,6-hexanediol starting from 3,4-dihydro-2H-pyran-2-carbaldehyde, which can be obtained from inexpensive feedstocks. The processes disclosed herein enable the production of 1,6-hexanediol under mild reaction conditions.

The 1,6-hexanediol obtained by the processes disclosed herein can be converted to industrially useful materials such as 1,6-diaminohexane. For example, 1,6-hexanediol can be reductively aminated to1,6-diaminohexane (1,6-hexanediamine) by methods known in the art. See, for example, U.S. Pat. No. 3,215,742; U.S. Pat. No. 3,268,588; and U.S. Pat. No. 3,270,059.

In some embodiments, the processes disclosed herein further comprise the steps:

(a) optionally, isolating at least a portion of the 1,6-hexanediol from the product mixture or second product mixture;

(b) contacting the 1,6-hexanediol with ammonia and hydrogen in the presence of a reductive amination catalyst at a temperature and for a time sufficient to form an amination product mixture comprising 1,6-diaminohexane; and

(c) optionally, isolating at least a portion of the 1,6-diaminohexane from the amination product mixture.

The reductive amination catalyst contains at least one element selected from Groups IB, VIB, VIIB, and VIII of the Periodic Table, for example iron, cobalt, nickel, ruthenium, rhodium, palladium, osmium, copper, chromium, iridium, or platinum. The elements may be in the zero oxidation state or in the form of a chemical compound. The reductive amination catalyst may be supported, unsupported or Raney-type. In one embodiment, the reductive amination catalyst contains ruthenium. In one embodiment, the reductive amination catalyst contains nickel. In one embodiment, the reductive amination catalyst is Raney nickel. In one embodiment, the reductive amination catalyst is Raney copper. In one embodiment, the reductive amination catalyst is Raney cobalt.

The reductive amination step is conducted by contacting 1,6-hexanediol, or a product mixture comprising 1,6-hexanediol, with ammonia and hydrogen in the presence of the catalyst for a time sufficient to form an amination product mixture comprising 1,6-diaminohexane. Useful temperatures for the reductive amination step can be in the range of about 40° C. to 300° C., for example in the range of about 75° C. to 150° C. Typically pressures can be in the range of about 2 MPa to 35 MPa, for example in the range of about 4 MPa to 12 MPa. The molar ratio of hydrogen to 1,6-hexanediol is typically equal to or greater than 1:1, for example in the range of 1:1 to 100:1, or in the range of 1:1 to 50:1.

The reductive amination step is typically performed in liquid ammonia solvent. The ammonia is used in stoichiometric excess with reference to 1,6-hexanediol. Typically, a molar ratio of 1:1 to 80:1 of ammonia to 1,6-hexanediol can be used, for example a molar ratio in the range of 10:1 to 50:1. Optionally, an additional solvent such as water, methanol, ethanol, butanol, pentanol, hexanol, an, ester, a hydrocarbon, tetrahydrofuran, or dioxane, can be used. The weight ratio of the additional solvent to 1,6-hexanediol is typically in the range of 0.1:1 to 5:1.

The reductive amination step can be performed in a fixed bed reactor or in a slurry reactor, for example a batch, continuous stirred tank reactor or bubble column reactor. The 1,6-diaminohexane may be isolated from the amination product mixture by any common methods known in the art, for example fractional distillation under moderate vacuum.

### EXAMPLES

The processes described herein are illustrated in the following examples. From the above discussion and these examples, one skilled in the art can ascertain the essential characteristics of the processes disclosed herein, and without departing from the spirit and scope thereof, can make various changes and modifications to adapt to various uses and conditions.

The following abbreviations are used in the examples: “° C.” means degrees Celsius; “Temp” means temperature; “wt %” means weight percent; “g” means gram; “mg” means milligram(s); “mL” means milliliter; “μL” means microliter; “mmol” means millimole “kPa” means kilopascals; “psi” means pounds per square inch; “h” means hour(s); “Ex” means Example, “Comp Ex” means Comparative Example; “Conv” means conversion.

Percent conversion and percent yield are defined as follows, where the mol of compounds are determined from calibrated gas chromatographic methods:

$$Conversion = \frac{100 * ( \begin{matrix}mol starting material charged - \\ mol starting material remaining\end{matrix} )}{mol starting material charged}$$

$$\% Yield = \frac{100 * mol product compound}{mol starting material charged}$$

### Materials

All commercial materials were used as received unless stated otherwise.

5% Ru/C catalyst was obtained from Sigma-Aldrich. CuOMnO₂Al₂O₃T-4489 was obtained from Suedchemie. The composition of this material was indicated to be 56 wt % CuO, 10 wt % MnO₂, and 34 wt % Al₂O₃. CuO/SiO₂ (BASF Cu-0860) was obtained from BASF Corporation. The composition of this material was indicated to be 25.0-40.0% copper, 10.0-20.0% silicon dioxide, 0.0-10.0% calcium oxide, 0.0-10.0% copper oxide, 0.0-7.0% Palygorskite 7, and 0.0-1.0% crystalline silica. Tetraammineplatinum (II) nitrate (catalog number 78-2010, lot number 20361000) was obtained from Strem. Para-ammonium tungsten oxide hydrate (stock number 22640, lot number 23449) was obtained from Alfa. NiCl₂×(7 to 8)H₂O, Cu(NO₃)₂×2.5 H₂O, Fe(NO₃)₃×9 H₂O, palladium chloride, and copper (II) chloride were obtained from Aldrich. Aerolyst 7708 TiO₂ (lot number 6/1837) was obtained from Evonik Industries. Zirconium(IV) oxide (catalog number 230693, lot number BCBG9965V) was obtained from Sigma-Aldrich. Silica gel 60 (catalog number 9385-3, lot number TA1598085) was obtained from EMD. Phosphotungstic acid H₃[P(W₃O₁₀)₄] was obtained from Sigma-Aldrich. D6310 carbon was obtained from BASF Corporation.

Tetrahydropyran-2-methanol (“THPM”, 98%), 3,4-dihydro-2H-pyran-2-methanol (“DHPM”, 98%), and tetrahydro-2H-pyran-2-carbaldehyde (“THPC”, 80%) were obtained from Sigma-Aldrich (St. Louis, Mo.). 3,4-Dihydro-2H-pyran-2-carbaldehyde (“ACD”, 90%) was obtained from Ivy Fine Chemicals (Cherry Hill, N.J.).

Methanol (“MeOH”, 99.5%), ethyl acetate (“EA”, 99.5%), and 2-propanol (“2-PrOH”, 99.5%) were obtained from EM Science. Ethanol (“EtOH”, 99.5%), 1-propanol (“1-PrOH”, 99.7%), 1-butanol (“1-BuOH”, 99.8%), 2-methylpropanol (“2MePrOH”, 99.0%), 1-hexanol (“HexOH”, 99.5%), tetrahydrofuran (“THF”, 99%), 1,4-dioxane (“dioxane”, 99%), dihexyl ether (“Hex₂O”, 97%), dibutyl ether (“Bu₂O”, 99%), hexyl hexanoate (“NH”, 97%), p-xylene (99%), and 1,6-hexanediol (99%) were obtained from Sigma-Aldrich.

### Purification of ACD

Before use, ACD was purified according to the following procedure. 2.5 Grams of ACD were added to 7.5 g of methanol and allowed to sit in an ice bath for about 2 hours. The mixture was filtered to remove solids. The solids were washed with 1-2 grams of cold methanol, the washings were combined with the filtrate, and the methanol was removed from the combined solution using a rotary evaporator to obtain purified ACD. Residual methanol was removed from the purified ACD by holding the material under vacuum for 4 hours. Purity of the purified ACD was found to be 92% by GC analysis. Purified ACD was stored at −10° C. and allowed to warm to room temperature before use.

### Catalyst Syntheses

A PtW/TiO₂ catalyst containing 4 wt % Pt and having a Pt/W weight ratio of 1:1 was prepared according to the following catalyst synthesis procedure. This catalyst is referred to herein as “4% PtW/TiO₂ (Pt/W 1:1)”. The other synthesized catalysts are named analogously herein.

18.41 Grams of Aerolyst 7708 TiO₂ (Evonik Industries, Lot #6/1837) that had been ground with a mortar and pestle and passed through a 0.0165 inch (420 micron) mesh sieve, was placed into a round bottomed flask and wetted with 18.0 mL of deionized water. To this was added 1.587 g of tetraammineplatinum (II) nitrate dissolved in 20.0 mL of deionized water. The flask was then placed onto a rotary evaporator and the material was allowed to mix while rotating for 15 minutes. Excess water was then removed under vacuum at 25° C. Subsequently, the flask was placed into a vacuum oven and dried overnight (17 h) at 110° C. After cooling to room temperature, the material was again wetted with 18.0 mL of deionized water. To this was then added 1.07 g of para-ammonium tungsten oxide hydrate dissolved in 60.0 mL of deionized water. The flask was then placed back onto a rotary evaporator and allowed to mix for 15 minutes. Excess water was then removed under vacuum at 25° C. The flask was then placed into a vacuum oven and dried overnight (17 h) at 110° C. After cooling to room temperature, the material was transferred to a ceramic boat and calcined in air at 400° C. for three hours. The final weight of the catalyst sample after calcining was 19.76 g.

Catalysts 2% PtW/TiO₂ (Pt/W 1:1), 1% PtW/TiO₂ (Pt/W 1:1), 1% PtW/TiO₂ (Pt/W 1:2), and 1% PtW/TiO₂(Pt/W 1:4), were prepared following the catalyst synthesis procedure disclosed herein above, except that appropriate amounts of Aerolyst 7708 TiO₂, tetraammineplatinum (II) nitrate and para-ammonium tungsten oxide hydrate were used to synthesize the target compositions.

Catalysts 4% NiW/TiO₂(Pt/W 1:1), PtFe/TiO₂ (Pt/Fe 1:1), 5% CuNi/TiO₂ (Cu/Ni 1:1), and 4% CuW//TiO₂ (Cu/W 1:1) were prepared following the catalyst synthesis procedure disclosed herein above except that appropriate amounts of tetraammineplatinum (II) nitrate, para-ammonium tungsten oxide hydrate, NiCl₂×(7 to 8)H₂O, Cu(NO₃)₂×2.5 H₂O, and Fe(NO₃)₃×9 H₂O, respectively, were used to synthesize the target compositions.

Catalysts 4% PtW/ZrO₂ (Pt/W 1:1) and 4% PtW/SiO₂ (Pt/W 1:1) were prepared following the catalyst synthesis procedure disclosed herein above except that the desired amount of ZrO₂ or SiO₂ was used instead of TiO₂.

Monometallic catalysts 4% Pt/TiO₂ and 4% W/TiO₂ were prepared following the catalyst synthesis procedure disclosed herein above except that only the first metal impregnation step was performed using appropriate amounts of Aerolyst 7708 TiO₂ and tetraammineplatinum (II) nitrate, or appropriate amounts of Aerolyst 7708 TiO₂ and para-ammonium tungsten oxide hydrate, respectively.

A catalyst referred to herein as 0.6% Pd-5.5% Cu on carbon was prepared under wet incipient conditions. The incipient wetness of D6310 carbon was determined to be 1.06 gram of water per gram of D6310 carbon. A solution of 0.100 g of palladium chloride, 1.565 g of copper (II) chloride, 9.6 g of water, and 1.0 g of 38% conc. HCI was added to 10 g of D6310 carbon that had been dried under nitrogen for 4 hours at 250° C. The mixture was mixed using a vortexer for about 15-20 seconds and then allowed to settle for 5 minutes. The mixing cycle was repeated 5 times until the entire liquid was absorbed by the carbon solids. The solids were then transferred onto a metal screen and allowed to air dry overnight. The material was then placed inside a quartz boat and, in a furnace, heated to and held at 125° C. for 4 hours, then subsequently heated to and held at 250° C. for 4 hours under nitrogen.

A catalyst referred to herein as (Cu/SiO₂)/(PWacid) 1:1 was prepared by physically mixing a supported copper oxide catalyst with a heteropoly acid according to the following procedure. About 1 g of dry supported copper oxide catalyst CuO/SiO₂ and 1 g of dry heteropoly acid H₃PW₁₂O₄₀ were mixed in a mortar and ground with a pestle for about 5 minutes. The mixture was then calcined in a furnace at 350° C. for about 1 hour before it was allowed to cool to room temperature. The catalyst was stored under nitrogen. The yield of catalyst was greater than 90% by weight.

TABLE 1
Descriptions and Compositions of Synthesized Catalysts

| Catalyst Description | Composition (Wt %) |
|---|---|
| 4% PtW/TiO₂(Pt/W 1:1) | 4% Pt, 4% W, 92% TiO₂ |
| 2% PtW/TiO₂(Pt/W1:1) | 2% Pt, 2% W, 94% TiO₂ |
| 1% PtW/TiO₂(Pt/W1:1) | 1% Pt, 1% W, 98% TiO₂ |
| 1% PtW/TiO₂(Pt/W1:2) | 1% Pt, 2% W, 97% TiO₂ |
| 1% PtW/TiO₂(Pt/W1:4) | 1% Pt, 4% W, 95% TiO₂ |
| 4% NiW/TiO₂(Pt/W1:1) | 4% Ni, 4% W, 92% TiO₂ |
| 4% PtFe/TiO₂ (Pt/Fe1:1) | 4% Pt, 4% Fe, 92% TiO₂ |
| 5% CuNi/TiO₂ (Cu/Ni 1:1) | 5% Pt, 5% Ni, 90% TiO₂ |
| 4% CuW//TiO₂ (Cu/W1:1) | 4% Cu, 4% W, 92% TiO₂ |
| 4% PtW/ZrO₂(Pt/W 1:1) | 4% Pt, 4% W, 92% ZrO₂ |
| 4% PtW/SiO₂(Pt/W 1:1) | 4% Pt, 4% W, 92% SiO₂ |
| 4% Pt/TiO₂ | 4% Pt, 96% TiO₂ |
| 4% W/TiO₂ | 4% W, 96% TiO₂ |
| 0.6% Pd—5.5% Cu on carbon | 0.6% Pd, 5.5% Cu, 93.9% C |
| (Cu/SiO₂)/(PWacid) 1:1 | 50% Cu/SiO₂, 50% |
| | H₃[P(W₃O₁₀)₄] |

### Examples 1 and 2

### Conversion of Acrolein Dimer (ACD) to 1,6-Hexanediol (16HD) and Comparative Examples A through F

### Non-Conversion of THPC and DHPM to 16HD

Examples 1 and 2 and Comparative Examples A through F were performed according to the following procedure.

In a glass vial equipped with a magnetic stir bar, 950 mg of solvent (ethanol or ethyl acetate [EA] as indicated in Table 2) were mixed with 50 mg of substrate as indicated in Table 2 and about 50 mg of 4% PtW/TiO_{2 (Pt/W} 1:1) catalyst were added. Each vial was capped with a perforated septum to limit vapor transfer rates. The vials were placed in a stainless steel (SS316) parallel pressure reactor (8 individual wells). The reactor was connected to a high pressure gas manifold and the contents were purged with nitrogen gas (1000 psi) three times before hydrogen was added. About 800 psi of hydrogen was added and the reactor was heated to 60° C. After 4 h the reactor was allowed to cool to room temperature and the pressure was released. Under an inert gas atmosphere, a 100 μL sample was taken from each vial, diluted with n-propanol, and analyzed by GC and GC/MS. Products were identified by matching retention times and mass spectra using authentic samples. Molar conversions of the substrates and product yields are given in Table 2.

TABLE 2
Results for Different Substrates at 60° C.

| | | | Conv | THPC | THPM | 16HD |
|---|---|---|---|---|---|---|
| Example | Substrate | Solvent | (mol %) | (mol %) | (mol %) | (mol %) |
| 1 | ACD | EA | 100 | 1 | 40 | 30 |
| 2 | ACD | EtOH | 100 | 0 | 37 | 23 |
| Comp. Ex. A | THPC | EA | 44 | 56 | 12 | 0 |
| Comp. Ex. B | THPC | EtOH | 33 | 67 | 4 | 0 |
| Comp. Ex. C | DHPM | EA | 100 | 3 | 85 | 0 |
| Comp. Ex. D | DHPM | EtOH | 100 | 0 | ~99 | 0 |
| Comp. Ex. E | THPM | EA | 0 | 0 | ~99 | 0 |
| Comp. Ex. F | THPM | EtOH | 0 | 0 | ~99 | 0 |

The results for Examples 1 and 2 show that ACD was converted to THPM and 1,6-hexanediol at 60° C., whereas the results for Comparative Examples A through F show that, when used as substrates, the corresponding saturated cyclic aldehyde THPC, the corresponding unsaturated cyclic alcohol DHPM, and the corresponding fully saturated cyclic alcohol THPM, were not converted to 1,6-hexanediol under the same reaction conditions. THPC was partially converted to other products, including the saturated analog THPM; DHPM was converted to THPM; and THPM appeared to be essentially unreactive under the conditions tested, as shown below in Scheme III.

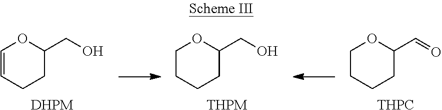

### Examples 3 through 18

### Conversion of ACD to 1,6-Hexanediol at 40° C. and 80° C. in Various Solvents

Examples 3 through 18 were performed according to the following procedure.

In a glass vial equipped with a magnetic stir bar were added 150 mg of ACD, 350 mg of solvent as indicated in Table 3, and about 150 mg of catalyst (4% PtW/TiO₂ (Pt/W 1:1). Each vial was capped with a perforated septum to limit vapor transfer rates. The vials were placed in a stainless steel (SS316) parallel pressure reactor (8 individual wells). The reactor was connected to a high pressure gas manifold and the contents were purged with nitrogen gas (1000 psi) three times before hydrogen was added. About 100 psi of hydrogen was added and the reactor was heated to the temperature indicated in Table 3. After 4 h the reactor was allowed to cool to room temperature and the pressure was released. Under an inert gas atmosphere, a 100 μL sample was taken from each vial, diluted with n-propanol, and analyzed by GC and GC/MS. Products were identified by matching retention times and mass spectra using authentic samples. For each Example, complete conversion of the ACD was observed. Molar product yields are given in Table 3.

TABLE 3
Product Yields for ACD Conversion in Various Solvents

| | Temp | | HexOH | THPM | 16HD |
|---|---|---|---|---|---|
| Example | (° C.) | Solvent | (mol %) | (mol %) | (mol %) |
| 3 | 80 | EtOH | 11 | 44 | 37 |
| 4 | 80 | HexOH | nd | 62 | 19 |
| 5 | 80 | Hex₂O | 1 | 76 | 5 |
| 6 | 80 | Bu₂O | 6 | 53 | 28 |
| 7 | 80 | HH | 3 | 67 | 10 |
| 8 | 80 | p-xylene | 2 | 69 | 16 |
| 9 | 80 | dioxane | 1 | 57 | 8 |
| 10 | 80 | THF | 2 | 78 | 12 |
| 11 | 80 | MeOH | 7 | 60 | 27 |
| 12 | 40 | EtOH | 11 | 38 | 28 |
| 13 | 40 | MeOH | 9 | 43 | 22 |
| 14 | 40 | 1-PrOH | 13 | 28 | 44 |
| 15 | 40 | 2-PrOH | 11 | 29 | 31 |
| 16 | 40 | 1-BuOH | 13 | 25 | 37 |
| 17 | 40 | 2MePrOH | 13 | 25 | 38 |
| 18 | 40 | HexOH | nd | 31 | 34 |

Note:
In Table 3, “nd” means not determined

Examples 3 through 18 show that ACD was converted to 1,6-hexanediol at 40° C. and 80° C. in a variety of solvents. Under the conditions tested, the highest molar yields of 1,6-hexanediol were obtained using ethano1,1-propanol, 1-butanol, or 2-methylpropanol as solvent. The highest molar yields of the fully saturated cyclic THPM were obtained using dihexyl ether, hexyl hexanoate, p-xylene, or THF as solvent at 80° C.

### Example 19

### Room Temperature Conversion of ACD to 1,6-Hexanediol

Example 19 shows the formation of a product mixture comprising 16HD, THPM, and HexOH from ACD at a 23 wt % substrate loading in 1-PrOH solvent at room temperature. The following procedure was used.

In a stainless steel (SS316) pressure reactor equipped with a magnetic stir bar 7.7 mL of 2-PrOH were added to 2.5 g (92% purity) of acrolein dimer (net 2.30 g, 20.5 mmol)) and about 2.5 g of 4% PtW/TiO₂ (Pt/W 1:1). The reactor was sealed and connected to a high pressure gas manifold and purged with nitrogen gas (1000 psi) three times before hydrogen was added. About 1000 psi of hydrogen was added and the reaction mixture was allowed to stir under pressure at about 25° C. After 4 h the reactor was depressurized. The reaction solution was diluted with 1-propanol and filtered through a standard 5 micron disposable filter. A sample was taken and analyzed by GC and GC/MS. Products were identified by matching retention times and mass spectra using authentic samples. Results for the reactor effluent are given in Table 4.

TABLE 4
Product Distribution for Example 19

| Example 19 | ACD | THPM | 16HD | HexOH | others | SUM |
|---|---|---|---|---|---|---|
| m [mg] | 0 | 670 | 983 | 365 | 220 | 2238 |
| n [mmol] | 0 | 6.0 | 8.3 | 3.6 | ~2.0 | ~19.9 |
| Yield | — | 29% | 41% | 18% | 10% | 98% |

### Examples 20 through 27

### Effect of Substrate Concentration on Conversion of ACD

Examples 20-27 show the effect of ACD concentration on the conversion to 1,6-hexanediol at 80° C. using EtOH or MeOH as solvent. The following procedure was used.

In each of eight glass vials equipped with a magnetic stir bar the desired amount of solvent (450 mg, 350 mg, 250 mg, or 150 mg) and ACD (50 mg, 150 mg, 250 mg, or 350 mg) were combined to form the desired solutions, and then equal parts of catalyst (4% PtW/TiO₂ (Pt/W 1:1) relative to ACD were added to each solution. The vials were capped with perforated septa to limit vapor transfer rates. The vials were placed in a stainless steel (SS316) parallel pressure reactor (8 individual wells). The reactor was connected to a high pressure gas manifold and the content was purged with nitrogen gas (1000 psi) three times before hydrogen was added. About 100 psi of hydrogen was added and the reactor was heated to 80° C. After 4 h the reactor was allowed to cool to room temperature and the pressure was released. Under inert gas atmosphere a 100 μL sample was taken from each vial, diluted with 1-propanol, and analyzed by GC and GC/MS. The ACD was converted quantitatively in all cases. Molar product yields are given in Table 5. Of the remaining material balance, approximately 75% consisted of acetals and hemiacetals of the starting aldehyde and approximately 25% consisted of non-detectable material (possibly polymers or high molecular weight acetals that were not amenable to analysis by gas chromatography).

TABLE 5
Product Yields with Different Loadings of ACD Substrate

| | Initial | | Catalyst | | | | Sum |
|---|---|---|---|---|---|---|---|
| Exam- | [ACD] | | Amount | HexOH | THPM | 16HD | (mol |
| ple | (wt %) | Solvent | (mg) | (mol %) | (mol %) | (mol %) | %) |
| 20 | 10 | EtOH | 50 | 15 | 46 | 34 | 95 |
| 21 | 30 | EtOH | 150 | 9 | 50 | 35 | 94 |
| 22 | 50 | EtOH | 250 | 6 | 44 | 24 | 74 |
| 23 | 70 | EtOH | 350 | 1 | 19 | 5 | 26 |
| 24 | 10 | MeOH | 50 | 9 | 57 | 25 | 91 |
| 25 | 30 | MeOH | 150 | 7 | 60 | 27 | 94 |
| 26 | 50 | MeOH | 250 | 5 | 48 | 19 | 71 |
| 27 | 70 | MeOH | 350 | 1 | 19 | 6 | 26 |

Under these reaction conditions, higher yields of 1,6-hexanediol were observed for initial ACD concentrations in the range of about 10 to 30 weight percent.

### Examples 28 and 29

### Comparative Examples G and H

### Product Distribution Using Different Catalyst Components as Catalyst

Examples 28 and 29 show the conversion of ACD to 1,6-hexanediol at 60° C. Comparative Examples G and H show the product distribution obtained from using a mono-metallic supported catalyst under the same conditions. Examples 28 and 29, and Comparative Examples G and H, were performed as follows.

In each of 4 glass vials equipped with a magnetic stir bar a solution of 150 mg ACD in 350 mg of 1-propanol was combined with 150 mg of the catalyst as indicated in Table 6. The vials were capped with perforated septa to limit vapor transfer rates. The vials were placed in a stainless steel (SS316) parallel pressure reactor (8 individual wells, 4 wells left empty). The reactor was connected to a high pressure gas manifold and the content was purged with nitrogen gas (1000 psi) three times before hydrogen was added. About 100 psi of hydrogen was added and the reactor was heated to 60° C. After 4 h the reactor was allowed to cool to room temperature and the pressure was released. Under an inert gas atmosphere, a 100 μL sample was taken from each vial, diluted with 1-propanol, and analyzed by GC and GC/MS. The ACD was converted quantitatively in all cases. In the case of Comparative Example H a mixture of acetals of acrolein dimer were observed. Molar product yields are given in Table 6.

TABLE 6
Product Yields with Different Catalyst Components as Catalyst

| Exam- | | HexOH | THPM | 16HD | Acetals | Sum |
|---|---|---|---|---|---|---|
| ple | Catalyst | (mol %) | (mol %) | (mol %) | (mol %) | (mol %) |
| 28 | 4% PtW/TiO₂ | 27 | 25 | 40 | 0 | 93 |
| 29 | 4% PtW/TiO₂ | 20 | 29 | 38 | 0 | 87 |
| Comp. | 4% Pt/TiO₂ | 3 | 66 | 3 | 0 | 72 |
| Ex. G | | | | | | |
| Comp. | 4% W/TiO₂ | 0 | 0 | 0 | >90 | 0 |
| Ex. H | | | | | | |

The results in Table 6 show that under the reaction conditions used, 1,6-hexanediol was formed from ACD when 4% PtW/TiO₂ (Pt/W 1:1) was used as catalyst. However, very little 1,6-hexanediol was formed under the same conditions when 4% Pt/TiO₂ was used as catalyst, and no 1,6-hexanediol was observed using 4% W/TiO₂ catalyst.

### Examples 30 through 45

### Conversion of ACD to 1,6-Hexanediol at 80° C. with Various Amounts of Water in the Solvent

Examples 30 through 45 demonstrate the formation of a product mixture comprising 1,6-hexanediol, 1-hexanol, and 1,2,6-hexanetriol from ACD at 80° C. in 1-PrOH or dioxane with various amounts of water present. These Examples were performed according to the following procedure.

In each of 8 glass vials equipped with a magnetic stir bar, 150 mg ACD and 350 mg of the mixture of water with 1-PrOH or with dioxane, as indicated in Table 7, was combined with 150 mg of 4% PtW/TiO₂(Pt/W 1:1). The vials were capped with perforated septa to limit vapor transfer rates. The vials were placed in a stainless steel (SS316) parallel pressure reactor (8 individual wells, 4 wells left empty). The reactor was connected to a high pressure gas manifold and the content was purged with nitrogen gas (1000 psi) three times before hydrogen was added. About 100 psi of hydrogen was added and the reactor was heated to 80° C. After 4 h the reactor was allowed to cool to room temperature and the pressure was released. Under inert gas atmosphere a 100 μL sample was taken from each vial, diluted with n-propanol, and analyzed by GC and GC/MS. The ACD was converted quantitatively in all cases. Molar product yields are given in Table 7.

TABLE 7
Product Yields for Examples 30 Through 45

| | | H₂O | | | | | |
|---|---|---|---|---|---|---|---|
| Exam- | | (wt | HexOH | THPM | 16HD | 126HT | Sum |
| ple | Solvent | %)* | (mol %) | (mol %) | (mol %) | (mol %) | (mol %) |
| 30 | 1-PrOH | 0 | 15 | 32 | 42 | 6 | 96 |
| 31 | 1-PrOH | 1 | 14 | 32 | 40 | 7 | 92 |
| 32 | 1-PrOH | 2 | 13 | 29 | 37 | 6 | 85 |
| 33 | 1-PrOH | 5 | 8 | 25 | 25 | 6 | 64 |
| 34 | 1-PrOH | 10 | 7 | 48 | 29 | 11 | 94 |
| 35 | 1-PrOH | 20 | 2 | 64 | 9 | 10 | 84 |
| 36 | 1-PrOH | 50 | 6 | 51 | 19 | 12 | 87 |
| 37 | 1-PrOH | 70 | 2 | 18 | 12 | 40 | 73 |
| 38 | Dioxane | 0 | 10 | 55 | 32 | 4 | 100 |
| 39 | Dioxane | 1 | 11 | 37 | 31 | 6 | 84 |
| 40 | Dioxane | 2 | 10 | 37 | 29 | 7 | 83 |
| 41 | Dioxane | 5 | 6 | 53 | 23 | 10 | 93 |
| 42 | Dioxane | 10 | 6 | 59 | 22 | 10 | 98 |
| 43 | Dioxane | 20 | 7 | 51 | 23 | 8 | 89 |
| 44 | Dioxane | 50 | 3 | 32 | 12 | 14 | 61 |
| 45 | Dioxane | 70 | 2 | 20 | 11 | 38 | 72 |

*Weight percent values were based on the total weight of solvent and water

The results in Table 7 indicate that under the reaction conditions employed, the yield of 1,6-hexanediol generally decreased with increasing amounts of water in the solvent. Larger amounts of water also resulted in less 1-hexanol formation. However, the highest yields of 126HT were observed at the highest water concentrations.

### Examples 46 through 58

### Comparative Examples J, K, and L

### Product Distribution from ACD Using Different Catalysts

Examples 46 through 58 show formation of a product mixture comprising 1,6-hexanediol from ACD at 80° C. using different catalysts. Examples 46 through 58 and Comparative Examples J, K, and L were performed according to the following procedure.

In each of 8 glass vials equipped with a magnetic stir bar, a solution of 50 mg ACD in 450 mg of EtOH was combined with 50 mg of the catalyst as indicated in Table 8. The vials were capped with perforated septa to limit vapor transfer rates. The vials were placed in a stainless steel (SS316) parallel pressure reactor (8 individual wells, 4 wells left empty). The reactor was connected to a high pressure gas manifold and the content was purged with nitrogen gas (1000 psi) three times before hydrogen was added. About 100 psi of hydrogen was added and the reactor was heated to 80° C. After 4 h the reactor was allowed to cool to room temperature and the pressure was released. Under an inert gas atmosphere, a 100 μL sample was taken from each vial, diluted with 1-propanol, and analyzed by GC and GC/MS. The ACD was converted quantitatively in each case. In the case of example 38 a mixture of acetals were observed. Molar product yields are given in Table 8.

TABLE 8
Product Yields from ACD at 80° C. Using Selected Catalysts

| | | HexOH | THPM | 16HD | 126HT | Sum |
|---|---|---|---|---|---|---|
| Example | Catalyst | (mol %) | (mol %) | (mol %) | (mol %) | (mol %) |
| 46 | 4% PtW/TiO₂ (Pt/W 1:1) | 17 | 42 | 31 | 1 | 91 |
| 47 | 2% PtW/TiO₂ (Pt/W 1:1) | 8 | 68 | 18 | 0 | 94 |
| 48 | 1% PtW/TiO₂ (Pt/W 1:1) | 0 | 91 | 3 | 0 | 95 |
| 49 | 1% PtW/TiO₂ (Pt/W 1:2) | 13 | 54 | 26 | 0 | 93 |
| 50 | 1% PtW/TiO₂ (Pt/W 1:4) | 3 | 81 | 10 | 0 | 94 |
| 51 | 4% NiW/TiO₂ (Pt/W 1:1) | 4 | 0 | 2 | 2 | 8 |
| Comp. | PtFe/TiO₂ (Pt/Fe 1:1) | 0 | 98 | 0 | 0 | 98 |
| Ex. J | | | | | | |
| Comp. | 5% Ru/C | 0 | 104 | 0 | 4 | 109 |
| Ex. K | | | | | | |
| 52 | 4% PtW/TiO₂ (Pt/W 1:1) | 16 | 44 | 36 | 1 | 97 |
| 53 | 5% CuNi/TiO₂ (Cu/Ni 1:1) | 0 | 85 | 1 | 0 | 86 |
| 54 | 4% CuW//TiO₂ (Cu/W 1:1) | 0 | 0 | 1 | 5 | 6 |
| 55 | 4% PtW/ZrO₂ (Pt/W 1:1) | 12 | 54 | 28 | 0 | 95 |
| 56 | 4% PtW/SiO₂ (Pt/W 1:1) | 3 | 85 | 9 | 0 | 98 |
| Comp. | (Cu/SiO₂)/(PWacid) 1:1 | 0 | 0 | 0 | 0 | 0 |
| Ex. L | | | | | | |
| 57 | 0.6% Pd-5.5% Cu on carbon | 0 | 0 | 1 | 0 | 1 |
| 58 | CuOMnO₂Al₂O₃ (T-4489) | 0 | 2 | 1 | 0 | 3 |

Under the reaction conditions used, formation of 1,6-hexanediol was observed for all catalysts except PtFe/TiO₂, 5% Ru/C, and (Cu/SiO₂)/(PWacid) 1:1. Highest yields of 1,6-hexanediol were obtained using 4% PtW/TiO₂ (Pt/W 1:1), 1% PtW/TiO₂ (Pt/W 1:2), and 4% PtW/ZrO₂ (Pt/W 1:1) catalysts.

What is claimed is:

1. A process comprising the step:
contacting 3,4-dihydro-2H-pyran-2-carbaldehyde, a solvent, and hydrogen in the presence of a catalyst at a reaction temperature between about 0° C. and about 120° C. at a pressure and for a reaction time sufficient to form a product mixture comprising 1,6-hexanediol.

2. The process of claim 1, wherein the solvent comprises an alcohol, an ether, an ester, an aromatic hydrocarbon, an aliphatic hydrocarbon, or mixtures thereof.

3. The process of claim 2, wherein the solvent is miscible with water and further comprises from about 0 weight percent to about 75 weight percent water, based on the total weight of water and solvent.

4. The process of claim 1, wherein the catalyst comprises a metal M1, a metal M2 or an oxide of M2, and a support, wherein:
M1 is Rh, Ir, Ni, Pd, or Pt, and M2 is Mo, W, or Re; or
M1 is Cu and M2 is Ni, Mn, or W.

5. The process of claim 4, wherein:
M1 is Cu and M2 is Ni, Mn, or W.

6. The process of claim 4, wherein:
M1 is Rh, Ir, Ni, Pd, or Pt, and M2 is Mo, W, or Re.

7. The process of claim 4, wherein M1 is Pt and M2 is W.

8. The process of claim 4, wherein the support comprises WO₃, V₂O₅, MoO₃, SiO₂, Al₂O₃, TiO₂, ZrO₂, tungstated ZrO₂, SiO₂—Al₂O₃, SiO₂—TiO₂, montmorillonite, zeolites, or mixtures thereof.

9. The process of claim 8, wherein M1 is Pt, M2 is W, and the support comprises TiO₂.

10. The process of claim 1, wherein the pressure is between about 690 kPa and about 6895 kPa.

11. The process of claim 1, wherein the concentration of 3,4-dihydro-2H-pyran-2-carbaldehyde in the solvent is between about 1 wt % and about 80 wt %, based on the total weight of 3,4-dihydro-2H-pyran-2-carbaldehyde and solvent.

12. The process of claim 1, wherein the contacting step comprises a first step of contacting the solvent and hydrogen in the presence of the catalyst to form an initial mixture, and a second step of adding the 3,4-dihydro-2H-pyran-2-carbaldehyde to the initial mixture.

13. The process of claim 1, wherein the contacting is performed in a continuous manner.

14. The process of claim 1, wherein the contacting is performed in a batch manner.

15. The process of claim 1, wherein the product mixture further comprises tetrahydro-2H-pyran-2-methanol.

16. The process of claim 1, wherein the product mixture further comprises 1,2,6-hexanetriol.

17. The process of claim 1, wherein the product mixture further comprises 1-hexanol.

18. The process of claim 1, further comprising a step of separating at least a portion of the 1,6-hexanediol from the product mixture.

19. The process of claim 1, wherein the 3,4-dihydro-2H-pyran-2-carbaldehyde is obtained from dimerization of acrolein.

20. The process of claim 1, wherein the product mixture further comprises tetrahydro-2H-pyran-2-methanol or 1,2,6-hexanetriol, and the process further comprises a step of:
reacting the product mixture with hydrogen in the presence of the catalyst at a second temperature between about 120° C. and about 260° C. at a second pressure of about 5515 kPa to about 13,800 kPa to form a second product mixture enriched in 1,6-hexanediol.

21. The process of claim 20, wherein the catalyst comprises a metal M1, a metal M2 or an oxide of M2, and a support, wherein:
M1 is Rh, Ir, Ni, Pd, or Pt, and M2 is Mo, W, or Re.

22. The process of claim 20, further comprising a step of separating at least a portion of the 1,6-hexanediol from the second product mixture.

23. The process of claim 1, or claim 20, further comprising the steps:
(a) optionally, isolating at least a portion of the 1,6-hexanediol from the product mixture or second product mixture;
(b) contacting the 1,6-hexanediol with ammonia and hydrogen in the presence of a reductive amination catalyst at a temperature and for a time sufficient to form an amination product mixture comprising 1,6-diaminohexane; and
(c) optionally, isolating at least a portion of the 1,6-diaminohexane from the amination product mixture.
